(19) United States
(12) Patent Application Publication
(10) Pub. No.: US 2015/0327444 A1
(43) Pub. Date: Nov. 19, 2015

(54) PROCESS FOR THE OVERPRODUCTION OF SHIKIMIC ACID AND PHENOLIC ACIDS IN FRUIT AND VEGETABLE CROPS

(71) Applicants: Instituto Tecnologico y de Estudios Superiores de Monterry, Monterrey (MX); The Texas A&M University System, College Station, TX (US)

(72) Inventors: Daniel Alberto Jacobo Velázquez, Monterrey (MX); Jorge Alejandro Benavides Lozano, Monterrey (MX); Luis Alberto Cisneros Zevallos, College Station, TX (US); Alejandro Becerra Moreno, Monterrey (MX)

(21) Appl. No.: 14/648,029

(22) PCT Filed: Dec. 19, 2013
(86) PCT No.: PCT/MX2013/000192

§ 371 Date: May 28, 2015

(30) Foreign Application Priority Data
Jan. 18, 2013 (MX) MX/A/2013/000789

(51) Int. Cl. A01G 1/00; A23L 1/212; A61K 36/23; A61K 31/191; A61K 31/05; A23L 1/29; A23L 3/3553

(52) U.S. Cl. CPC A01G 1/001; A23L 1/29; A23L 1/2121; A23L 3/3553; A61K 31/191; A61K 31/05; A61K 36/23; A23V 2002/00

(57) ABSTRACT

The invention relates to a process for the overproduction of shikimic acid and phenolic compounds in fruit and vegetable crops, by means of the combined post-harvest application of abiotic stresses and glyphosate to fruit and vegetable crops in order to produce bioactive compounds of wide-ranging interest and commercial value. The carrot (Daucus carota) was used as the fruit and vegetable crop model. The process can be used to produce and store shikimic acid (AS) and a wide variety of phenolic compounds (CF) in the treated fruit and vegetable crop. There was an increase of more than 1000% in the concentration of shikimic acid and other compounds overproduced and stored as a result of the application of this technology in relation to concentrations in untreated fruit and vegetable crops. The stressed fruit and vegetable crops can be subsequently processed in order to extract and purify the bioactive compounds of interest.

### OBJECT OF THE INVENTION

This invention relates to the development of a process for the overproduction of bioactive compounds such as shikimic acid (SA) and phenolic compounds (PC) in horticultural crops through the application of post-harvesting abiotic stresses

### BACKGROUND

In recent years the incidence of chronic-degenerative diseases and pandemics in the general population has increased significantly. As a result there is growing interest in all those fields (identification, production, recovery, etc.) related to the study of bioactive compounds with pharmaceutical and/or nutraceutical applications. The use of plants for the production of chemo-preventive compounds having high commercial value has been one of the most utilized strategies. Genetic and metabolic engineering have been used to generate crops which overproduce compounds having pharmaceutical and nutraceutical applications. Many crops, together with other expression systems, both eukaryote [Ye, X., Al-Babili, S., Klöti, A., Zhang, J., Lucca, P., Beyer, P., Potrykus I. Engineering the provitamin A (beta-411 carotene) biosynthetic pathway into (carotenoid-free) rice endosperm. Science. 2000, 287, 303-305], [Niggeweg, R., Michael, A. J., Martin, C. Engineering plants with increased levels of the antioxidant chlorogenic acid, Nat. Biotechnol. 2004, 22, 746-754. 351] and prokaryote [Johansson, L., Lindskog, A., Silfversparre, G., Cimander, C.; Nielsen, K. F., Lidén, G. Shikimic acid production by a modified strain of E-coli (W3110.shikl) under phosphate-limited and carbon-limited conditions. Biotechnol. Bioen. 2005, 92, 541-552] [Diretto, G., Al-Babili, S., Tavazza, R., Papacchioli, V., Beyer, P., Giuliano, G. Metabolic engineering of potato carotenoid content through tuber-specific over expression of a bacterial mini-pathway. PLoS ONE. 2007, 2, e350] have been genetically modified to encourage the production and accumulation of metabolites of commercial interest. Nevertheless metabolic engineering is technically complex and the commercial scale cultivation of transgenic plant lines has on many occasions been questioned because of potential risks to the environment and human health [Colwell, R. K., Norse, E. A., Pimentel, D., Sharples, F. E., Simberloff, D. Genetic engineering in agriculture. Science. 1985, 229, 111-112]. It is because of this that genetically modified products have given rise to an enormous public debate concerning their legal status, their positioning in the environment, their social acceptance and ethical questions. The public's general perception of the use of these transgenic plant lines has been emphatically charged with disapproval because the introduction of foreign genes into plants can give rise to the production of substances that are harmful to human health, thus affecting the surrounding environment through variations in the same species and the loss of great genetic diversity [Kariyawasam, K. Legal liability, intellectual property and genetically modified crops: their impact on world agriculture. Pac. Rim L. & Policy J. 2010, 19, 459-485].

The application of post-harvesting abiotic stresses (wounding, the use of controlled and modified atmospheres, gassing with ethylene (C₂H₄) for ripening, varying storage temperature and desiccation, etc.) is a practical and efficient technology which makes it possible to accumulate antioxidant metabolites in horticultural crops [Cisneros-Zevallos, L. The use of controlled post-harvest abiotic stresses as a tool for enhancing the nutraceutical content and adding value of fresh fruits and vegetables. J. Food Sci. 2003, 68, 1560-1565] [Toivonen, P. M. A., Hodges, D. M., Abiotic stress in harvested fruits and vegetables. In: Abiotic stress in plants—mechanisms and adaptations. Shanker, A., Venkateswarlu, B. (Eds.), 2011, 39-58]. For example the production and accumulation of high levels of caffeoylquinic acids have been promoted in carrots treated with wounding stress alone [Surjadinata, B. B., Cisneros-Zevallos, L. Biosynthesis of phenolic antioxidants in carrot tissue increases with wounding intensity. Food Chem. 2012, 134, 615-624], or in combination with other abiotic stresses, such as UV ultraviolet radiation [Surjadinata, B. B. Wounding and Ultraviolet Radiation Stresses Affect the Phenolic Profile and Antioxidant Capacity of Carrot Tissue. Ph.D. dissertation, Texas A&M University, College Station, Tex., 2006], hyperoxia [Jacobo-Velázquez, D. A., Martínez-Hernández, G. B., Rodríguez, S., Cao, C.-M., Cisneros-Zevallos, L. Plants as biofactories: Physiological role of reactive oxygen species on the accumulation of phenolic antioxidants in carrot tissue under wounding and hyperoxia stress. J. Agric. Food Chem. 2011, 59, 6583-6593], the application of plant hormones [Heredia, J. B., Cisneros-Zevallos, L. The effect of exogenous ethylene and methyl jasmonate on pal activity, phenolic profiles and antioxidant capacity of carrots (Daucus carota) under different wounding intensities. Post-harvest Biol. Technol. 2009, 51, 242-249], [Heredia, J. B., Cisneros-Zevallos, L. The effect of exogenous ethylene and methyl jasmonate on the accumulation of phenolic antioxidants in selected whole and wounded fresh produce. Food. Chem. 2009, 115, 1500-1508], and the application of enzyme inhibitors such as herbicides [Becerra-Moreno, A., Benavides, J., Cisneros-Zevallos, L., Jacobo-Velázquez, D. A. Plants as biofactories: glyphosate-induced production of Shikimic acid and phenolic antioxidants in wounded carrot tissue J. Agric. Food Chem. 2012, 60, 11378-11386]. Caffeoylquinic acids are phenolic compounds (PC) with great potential for the prevention and treatment of different degenerative diseases such as HIV [Robinson, W. E., Jr., Cordeiro, M., Abdel-Malek, S., Jia, Q., Chow, S. A., Reinecke, M. G., Mitchell, W. M. Dicaffeoylquinic acid inhibitors of human immunodeficiency virus integrase: inhibition of the core catalytic domain of human immunodeficiency virus integrase. Mol. Pharmacol. 1996, 50, 845-855], [Zhu, K., Cordeiro, M. L., Atienza, J., Robinson, W. E., Jr., Chow, S. A. Irreversible inhibition of human immunodeficiency virus type 1 integrase by dicaffeoylquinic acids. J. Virol. 1999, 73, 3309-3316], Alzheimer's disease [Kim, S.-S., Park, R.-Y., Jeon, H.-J., Kwon, Y.-S., Chun, W. Neuroprotective effect of 3,5-dicaffeoylquinic acid on hydrogen peroxide-induced cell death in SH-SY5Y cells. Phytother. Res. 2005, 19, 243-245], obesity [Thorn, E. The effect of chlorogenic acid enriched coffee on glucose absorption in healthy volunteers and its effect on body mass when used long-term in overweight and obese people. J. Int. Med Res. 2007, 35, 900-908] and hepatitis B [Wang, G.-F., Shi, L.-P., Ren, Y.-D., Liu, Q.-F., Liu, H.-.F., Zhang, R.-J., Li, Z., Zhu, F.-H., He, P.-L., Tang, W., Tao, P.-Z., Li, C; Zhao, W.-M., Zuo, J.-P. Anti-hepatitis B virus activity of chlorogenic acid, quinic acid and caffeic acid in vitro and in vivo. Antivir. Res. 2009, 83, 186-190].

In lettuce and carrots the accumulation of phenolic compounds through the application of ethylene has been reported [Ke, D., Saltveit M. Plant hormone interaction and phenolic metabolism in the regulation of russet spotting in iceberg lettuce. Plant. Physiol. 1988, 88, 1136-1140] [Lafuente, T., Lopez-Galvez, G., Cantwell, M., Fa Yang, S. Factors influencing ethylene-induced isocoumarin formation and increased respiration in carrots. J. Am. Soc. Hortic. Sci. 1996, 121, 537-542]. It has also been reported that the application of UVB light and light produced by fluorescent tubes to potatoes [Percival, G., Baird, L. Influence of storage upon light-induced chlorogenic acid accumulation in potato tubers (Solanum tuberosum L.) J. Agric. Food Chem. 2000, 48, 2476-2482], cabbages [Craker, L., Wetherbee, P. Ethylene, light and anthocyanin synthesis. Plant Physiol. 1973, 51, 436-438] and apples [Reay, P. The role of low temperatures in the development of the red blush on apple fruit (Granny Smith). Sci. Hortic. 1999, 79, 113-119] gives rise to the accumulation of chlorogenic acid, anthocyanins and quercetin respectively.

The biosynthesis of secondary metabolites induced by the application of various post-harvest abiotic stresses in fruits and vegetables has been studied in recent years. However the effect of wounding stress on the accumulation of primary metabolites has not been completely characterized. Wounding stress activates metabolic pathways related to metabolite synthesis [Dyer, W. E., Henstrand, J. M., Handa, A. K., Herrmann, K. M. Wounding induces the first enzyme of the shikimate pathway in Solanaceae. Proc. Natl. Acad Sci. U.S.A. 1989, 86, 7370-7373], [Sharma, R., Jain, M., Bhatnagar, R. K., Bhalla-Sarin, N. Differential expression of DAHP synthase and chorismate mutase in various organs of Brassica juncea and the effect of external factors on enzyme activity. Physiol. Plant. 1999, 105, 739-745], [Jacobo-Velázquez, D. A., Cisneros-Zevallos, L. An alternative use of horticultural crops: stressed plants as biofactories of bioactive phenolic compounds. Agriculture. 2012, 2, 259-271]. A primary metabolite which accumulates in plants as a response to wounding stress is shikimic acid (SA) [Becerra-Moreno, A., Benavides, J., Cisneros-Zevallos, L., Jacobo-Velázquez, D. Plants as biofactories: glyphosate-induced production of shikimic acid and phenolic antioxidants in wounded carrot tissue, J. Agric. Food Chem. 2012, 60, 11378-11386]. This compound is of great value in the pharmaceutical industry because it is used in the production of Oseltamivir (Tamiflu®). This product is used as a first line of defense in the treatment of influenza [Farina, V., Brown, J. D. Tamiflu: the supply problem. Angew. Chem., Int. Ed. 2006, 45, 7330-7334]. The main natural sources of SA are plants of the Illicium genus, such as Chinese Star Anise [Bochkov, D. V., Sysolyatin, S. V., Kalashnikov, A. I., Surmacheva, I. A. Shikimic acid: review of its analytical, isolation, and purification techniques from plant and microbial sources. J. Chem. Biol. 2012, 5, 5-17] and, although there are other methods for producing and extracting SA using microorganisms, none of these are at the moment as economically viable as star anise. This variety of anise is only available in 4 provinces in China which have the necessary conditions for its growth and it can only be harvested once a year, around the month of May, [Payne, R., Edmonds, M. Isolation of shikimic acid from star anise, J. Chem. Educ. 2005, 82, 599-600], [von Itzstein M. The war against influenza: discovery and developments on sialidase inhibitors. Nat. Rev. Drug Discov. 2007, 6, 967-974]; other attempts to produce this plant under other conditions have resulted in a smaller yield of SA.

However because star anise is a product consumed by human beings in China the government of that country controls the quantity that can be exported and limits it to a particular percentage of total production. As the availability of natural sources rich in SA is insufficient to supply world demand for this compound, given latent influenza pandemics [Farina, V., Brown, J. D. Tamiflu: the supply problem. Angew. Chem., Int. Ed. 2006, 45, 7330-7334] there is therefore a need to investigate additional sources of SA.

Although the application of wounding stress activates metabolic pathways related to the synthesis of SA in plants, it is to be expected that the tissue will accumulate a low concentration of this metabolite because it is used in subsequent metabolic reactions for production of the aromatic amino acids required for the biosynthesis of secondary metabolites [Davis, B. D. Aromatic biosynthesis. I. The role of shikimic acid. J. Biol. Chem. 1951, 191, 315-325]. Thus it would be useful to develop a strategy to reduce the rate at which SA due to a wounding stress effect is used up, thus making it possible to accumulate it in horticultural crops. As part of this research work the use of glyphosate (N-phosphonomethyl glycine) in combination with abiotic stress (wounding stress in particular) has been envisaged as a way of inducing the production and accumulation of SA. When applied to plants, glyphosate inhibits the biosynthesis of aromatic amino acids, blocking the action of the enzyme 5-enolpyruvylshikimate-3-phosphate synthase (EPSP synthase) [Amrhein, N., Deus, B., Gehrke, P., Steinrücken, H. C. The site of the inhibition of the shikimate pathway by glyphosate. II. Interference of glyphosate with chorismate formation in vivo and in vitro. Plant Physiol. 1980, 66, 830-834].

Shikimic acid 3-phosphate (S3F) is the substrate for EPSP synthase, and when this enzyme is inhibited by glyphosate the S3F is not used and is quickly converted into SA [Harring, T., Streibig, J. C; Husted, S. Accumulation of shikimic acid: a technique for screening glyphosate efficacy. J. Agric. Food Chem. 1998, 46, 4406-4412]. Protocols for the accumulation of SA in different plant materials using glyphosate have been reported. For example, Anderson (2012 U.S. Pat. No. 8,203,020) reports a method for the accumulation of SA in alfalfa and wheat in which first of all the plants are grown in the absence of glyphosate for a particular time, and are then afterwards treated with the same for a second period of time, during which the plant increases its SA levels. Finally the plant is harvested and is ready for a subsequent process for the recovery and purification of SA. Bresnahan, G. A., Manthey, F. A., Howatt, K. A. and Chakraborty, M. [Glyphosate applied pre-harvest induces shikimic acid accumulation in hard red spring wheat Triticum aestivum. J. Agric. Food Chem. 2003, 51, 44-47] have evaluated the accumulation and distribution of SA through the various tissues in wheat plants treated with glyphosate, finding high concentrations of the compound. The maximum SA concentration occurred 3-7 days after treatment with glyphosate and subsequently decreased until the plant was harvested. Zelaya, I. A., Anderson, J. A., Owen, M. D. and Landes, R. D. [Evaluation of spectrophotometric and HPLC methods for shikimic acid determination in plants: models in glyphosate-resistant and susceptible crops. J. Agric. Food Chem. 2011, 59, 2202-2212] describe a similar process in maize and soya plants, where the SA concentration increased in the plant for a certain time after application of the herbicide.

The pre-harvest application of glyphosate together with the change in SA levels in different plant materials has also been used as a technique for monitoring crops which are and are not resistant to the herbicide. For example [Pline, W. A., Wilcut, J. W., Duke, S. O., Edmisten, K. L. and Wells, R. [Tolerance and accumulation of shikimic acid in response to glyphosate applications in glyphosate-resistant and non-glyphosate-resistant cotton (Gossypium hirsutum L.) J. Agric. Food Chem. 2002, 50, 506-512] propose that the increase in SA levels in response to glyphosate inhibition is a quick and precise means for measuring the damage induced in non-resistant plants through the action of that herbicide. In their results they demonstrate that SA does not accumulate in the leaves of cotton (Gossypium hirsutum) which is resistant to the herbicide in response to treatments with glyphosate, but it does increase significantly in all the tissues of non-resistant cotton. Harring, T., Streibig, J. C; Husted, S. [Accumulation of shikimic acid: a technique for screening glyphosate efficacy. J. Agric. Food Chem. 1998, 46, 4406-4412] established that the accumulation of SA found in the leaves of the rape plant (Brassica napus L. cv. Iris) is Related to the applied dose of glyphosate, even 5 hours after treatment. Likewise Anderson, K. A., Cobb, W. T. and Loper, B. R. [Analytical method for determination of shikimic acid: shikimic acid proportional to glyphosate application rates. Commun. Soil Sci. Plant Anal. 2001, 32(17/18), 2831-2840] found that SA is directly proportional to the levels of application of glyphosate herbicide in wheat tissue.

At the present time various protocols have been reported for inducing the accumulation of SA and other compounds having high nutraceutical value produced in various plant materials. Most of these protocols are complicated due to the fact that they take place during a stage of pre-harvesting of the plant, which implies long waiting times and low yields. Consequently the potential scaling up of agricultural procedures of this kind has been perceived negatively from the economic point of view.

In order to minimize the disadvantages attributed to established protocols for the accumulation of SA and PC in plant systems the use of alternative techniques, such as the modification of prokaryote organisms [Farina, V., Brown, J. D. Tamiflu: the supply problem. Angew. Chem., Int. Ed. 2006, 45, 7330-7334] and chemical synthesis of the compounds [Fukuta, Y., Mita, T., Fukuda, N., Kanai, M., Shibasaki, M. De novo synthesis of Tamiflu via a catalytic asymmetric ring-opening of meso-aziridines with TMSN3. J. Am. Chem. Soc. 2006, 128, 6312-6313] [Yeung, Y. Y., Hong, S. S., Corey, E. J. A short enantioselective pathway for the synthesis of the anti-Influenza neuramidase inhibitor oseltamivir from 1,3-butadiene and acrylic acid. J. Am. Chem. Soc. 2006, 128, 6310-6311] have been proposed, but the latter is inefficient and very complex for scaling up to an industrial level [Draths, K. M., Knop, D. R., Frost, J. W. Shikimic acid and quinic acid: replacing isolation from plant sources with recombinant microbial biocatalysis. J. Am. Chem. Soc. 1999, 121, 1603-1604]. Frost et al. (2002 U.S. Pat. No. 6,472,169 and 2003 U.S. Pat. No. 6,613,552) report the building of a strain of E. coli having various genetic modifications for the production of SA. Together all the genetic modifications made increase the flow of metabolites to SA, thus increasing its concentration. However because of these same genetic modifications this strain is incapable of synthesizing the aromatic amino acids tryptophan, phenylalanine and tyrosine, together with other essential compounds (p-hydroxybenzoic acid, p-aminobenzoic acid and 2,3-dihydroxybenzoic acid).

In order to be able to grow this strain in a fermenter the fermentation medium must be supplemented with these 6 essential components. It is also important to bear in mind that other contaminants, such as quinic acid, are also produced in excess in this fermentation, which means that it is necessary to use costly and/or difficult techniques for scaling up during the recovery and purification process [Draths, K. M., Knop, D. R., Frost, J. W. Shikimic acid and quinic acid: replacing isolation from plant sources with recombinant microbial biocatalysis. J. Am. Chem. Soc. 1999, 121, 1603-1604]. Improvements in this strain have been reported, and these have mainly focused on improving the yield of SA during fermentation [Knop, D. R., Draths, K. M., Chandran, S. S., Barker, J. L., Frost, J. W. Hydroaromatic equilibrium during biosynthesis of shikimic acid. J. Am. Chem. Soc. 2001, 123, 10173-10182], [Bongaerts, J., Krämer, M., Müller, U., Raeven, L., Wubbolts, M. Metabolic engineering for microbial production of aromatic amino acids and derived compounds. Metab. Eng. 2001, 3, 289-300], [Chandran, S. S., Yi, J., Draths, K. M., Von Daeniken, R., Weber, W., Frost, J. W. Phosphoenolpyruvate availability and the biosynthesis of shikimic acid. Biotechnol Prog. 2003, 19, 808-814], [Krämer, M., Bongaerts, J., Bovenberg, R., Kremer, S., Müller, U., Orf, S., Wubbolts, M., Raeven, L. Metabolic engineering for microbial production of shikimic acid. Metab Eng. 2003, 5, 277-283]. Similar work has been carried out on other prokaryote organisms. For example Iomantas et al. (2002 U.S. Pat. No. 6,436,664) describe a method for the production of SA using modified strains of Bacillus subtilis which are deficient in shikimate kinase and 5-enolpyruvylshikimate-3-phosphate synthase activity, which produce and accumulate SA in the culture medium. Shirai et al. (2001 European Patent Application EP1092766) report a similar process with a mutant strain of Citrobacter freundii for producing SA through fermentation.

A different focus for fermentations has been reported by Bogosian et al. (2011 US Patent Application US20110020885) who developed a method for producing SA which includes fermentation of a recombinant strain of E. coli in which glyphosate is added to the culture medium as an inhibitor of EPSP synthase. At the present time these fermentations fulfill 30% of world demand for SA [Farina, V., Brown, J. D. Tamiflu: the supply problem. Angew. Chem., Int. Ed. 2006, 45, 7330-7334]. However at the present time the yields of SA obtained in prokaryote systems are low, implying that complex stages are used to purify the compound.

Although some of the processes mentioned above use glyphosate to induce the accumulation of SA, in none of these is glyphosate applied to horticultural crops at a post-harvest stage. Thus, as part of this research, it has been established that the use of post-harvest abiotic stresses and glyphosate in horticultural crops is an attractive alternative to intensive agriculture and genetic engineering to produce bioactive compounds, not limited to SA, but also covering other families of compounds having proven nutraceutical and pharmaceutical capabilities. Although the potential use of abiotic stresses to produce secondary metabolites such as phenolic compounds has been reported in the literature [Jacobo-Velázquez, D. A., Martínez-Hernández, G. B., Rodríguez, S., Cao, C.-M; Cisneros-Zevallos, L. Plants as biofactories: Physiological role of reactive oxygen species on the accumulation of phenolic antioxidants in carrot tissue under wounding and hyperoxia stress. J. Agric. Food Chem. 2011, 59, 6583-6593] the accumulation of SA (primary metabolite) in horticultural crops by abiotic stresses has only been marginally characterized. Likewise, the use of glyphosate (generally used as a herbicide) as a modulator for the flow of carbon from primary metabolism to secondary metabolism, in order to induce the selective accumulation of SA and PC of interest in previously stressed tissues, has recently been studied and characterized by our research group [Becerra-Moreno, A., Benavides, J., Cisneros-Zevallos, L., Jacobo-Velázquez, D. Plants as biofactories: glyphosate-induced production of shikimic acid and phenolic antioxidants in wounded carrot tissue. J. Agric. Food Chem. 2012, 60, 11378-11386]. As part of the development of this technology carrots (Daucus carota) have been selected as a plant model, as they are a crop that is widely distributed throughout the world and is easy to handle. Today carrots are one of the most widely produced vegetable crops in the world. Around 340,000 tons/year of carrots are grown in Mexico [FAOSTAT. 2010. FAO Statistical Databases. Agricultural Data]. However when post-harvest practices are inadequate, quality standards are compromised, which means that some of the output has to be rejected (˜10% of annual production in Mexico). In this study by our research group, whose invention is presented and described in this document, it has been found that the combination of post-harvest abiotic stress and the application of glyphosate have a significant effect on the accumulation of SA and also other nutraceutical compounds of great commercial interest. Spraying a concentrated solution of glyphosate onto carrot tissue which has been stressed by wounding increases the SA and chlorogenic acid (CA) concentrations by more than 1000 and 5000% respectively. Thus, given growing world demand, investigating alternative uses for these carrots, regarded as waste, as biofactories for the production of plant chemicals having high pharmaceutical and nutraceutical value is of vital importance for the development of alternative technologies for the production of SA and PC. Although carrots have been used as the model plant system for developing this technology, it is to be hoped that the technical result that would be obtained with other types of horticultural crops would be similar, bearing in mind of course that the yields and profiles of nutraceutical compounds synthesized and accumulated would have to be characterized individually. Likewise, the application of a post-harvest abiotic stress (other than wounding) or the combination of various abiotic stresses, together with glyphosate, could give rise to a similar technical result in different plant models. Also, in the case of carrots, it has been found that the application of wounding stress alone makes it possible to obtain a significant concentration of SA in the horticultural crop 24 hours after application of that stress. This has not been reported by any author. Despite this, as already mentioned, the concentration of this compound is very much greater when wounding stress is used jointly with the application of glyphosate.

### BRIEF DESCRIPTION OF THE FIGURES

FIG. 1. Diagrammatical representation of the proposed process for the overproduction of bioactive compounds in horticultural crops.

FIG. 1b. Diagrammatical representation of the proposed process for the overproduction of bioactive compounds in horticultural crops, including a drying stage.

FIG. 1c. Diagrammatical representation of the proposed process for the overproduction of bioactive compounds in horticultural crops, including a drying and grinding stage.

FIG. 1d. Diagrammatical representation of the proposed process for the overproduction of bioactive compounds in horticultural crops, including a stage of glyphosate application.

FIG. 1e. Diagrammatical representation of the proposed process for the overproduction of bioactive compounds in horticultural crops, including a stage of glyphosate application and drying.

FIG. 1f. Diagrammatical representation of the proposed process for the overproduction of bioactive compounds in horticultural crops, including a stage of glyphosate application, drying and grinding.

FIG. 1g. Diagrammatical representation of the proposed process for the overproduction of bioactive compounds in horticultural crops, including a stage of glyphosate application, drying and subsequent stages of recovery, purification and cleaning.

FIG. 2. Accumulation of shikimic acid during the storage of grated carrots. The samples were stored for 48 hours at 25° C. under atmospheric pressure with a relative humidity of 65% in total darkness. The values represent the mean of 4 repetitions with their standard error bars. Data with different letters indicate statistically significant differences in the LSD test (p<0.05).

FIG. 3. Effect of the method of applying concentrated glyphosate solution on the concentration of bioactive compounds in a carrot crop (submerged and sprayed).

FIG. 4. HPLC-PDA chromatograms at 320 nm (A), 280 nm (B) and 215 nm (C). (a) grated carrot before storage (0 h), (b) grated carrot stored for 24 hours at 25° C., (c) grated carrot sprayed with glyphosate and stored for 24 hours at 25° C. Tentative identification of the chromatographic peaks was carried out as indicated in Table 2. Assigned peaks include: (1) shikimic acid, (2) protocatechuic acid, (3) a gallic acid derivative, (4) chlorogenic acid, (5) 3,5-dicaffeoylquinic acid, (6) p-coumaric acid derivative A, (7) 4,5-dicaffeoylquinic acid, (8) p-coumaric acid, (9) ferulic acid, (10) p-coumaric acid derivative B, (11) ferulic acid derivative, (12) isocoumarin.

### DETAILED DESCRIPTION OF THE INVENTION

This patent application describes a novel process for inducing the overproduction and accumulation of bioactive compounds such as shikimic acid (SA) and phenolic compounds (PC) in plants whose fruits, seeds, leaves, stems and roots are edible (horticultural crops) through the application of post-harvest abiotic stresses to at least one of the parts of the abovementioned horticultural crops, in which this process is potentiated through the addition of glyphosate in one of its stages.

This patent application comprises providing a process for the overproduction of shikimic acid and phenolic compounds in a horticultural crop basically in 2 stages after harvesting of the horticultural crop. The plant material may originate from various industries, being of easy access, favoring its exhaustive manipulation at both laboratory and industrial level. The plant material discarded by various industrial sectors may arise from the presence of some factors during growth and maturation of the same which can adversely affect them and thus reduce their commercial value as a raw material for human consumption [Pantastico, E. B. Post-harvest physiology, handling and utilization of tropical and subtropical fruits and vegetables. Avi Pub. Co. 1975], [Koda, Y., Okazawa, Y. Influences of environmental, hormonal and nutritional factors on potato tuberization in vitro. Jap. J. Crop Sci. 1983, 52, 582-591] [Salunkhe, D. K., Kadam, S. S. Treatise on the science and technology of horticultural crops. Production, composition, storage and processing. Editorial Acribia; Translation by Orlando Pablo Väzquez Yäñez and Pilar Calo Nata. 2004]. Some of the most common factors in different horticultural crops which reduce their quality during growth are shown in Table 1, taking as examples two of the most common crops worldwide, carrots and potatoes.

TABLE 1
Most common factors in some horticultural
crops during growth and ripening, which reduce their
quality and commercial value as raw materials for human
consumption.

| Horticultural | |
|---|---|
| crop | Factors |
| Carrots | Wounding due to frost and hail (<−1.5° C.). |
| | Absence of bright orange color, |
| | soft texture, firmness, sweet |
| | flavor and the presence of a |
| | fibrous center. |
| | Deformed and bifurcated roots. |
| | Presence of a green heart and |
| | shoulders caused by exposure to |
| | sunlight. |
| Potatoes | Wounding due to frost and hail (<−1.5° C.). |
| | Short growth period. |
| | Fungal diseases (Phythopthora |
| | infestans) and bacterial diseases |
| | (Erwinia sp.) |
| | Insufficient tuber size. |
| | Presence of internal and external |
| | green coloration. |

The process to which this patent application relates provides added value to horticultural crops, especially when these have been discarded as they do not comply with the quality standards of various industrial/commercial sectors.

Thus this process can be applied to plant material of very different nature [all plants whose fruits, seeds, leaves, stems or roots are edible. These include roots (carrots, radishes, among others), bulbs or tubers (potatoes, onions, garlic, to mention some), flowers and inflorescences (broccoli, cauliflower, artichokes, among others), fruits and seeds (apples, cucumbers, eggplant, among others), leaves (kale, lettuce, cabbage, among others), and pulses (kidney beans, chickpeas, peas, among others)] in any quality available (high quality, rejects, waste, bagasse, among others).

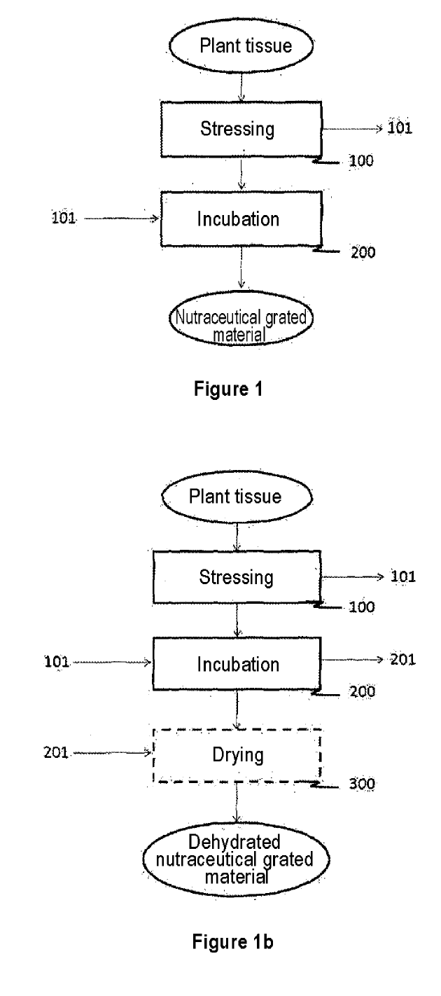

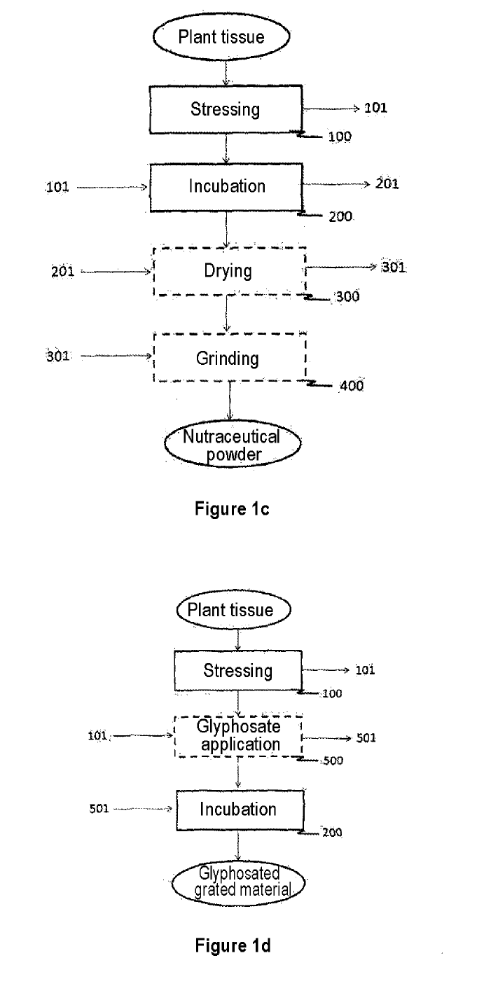

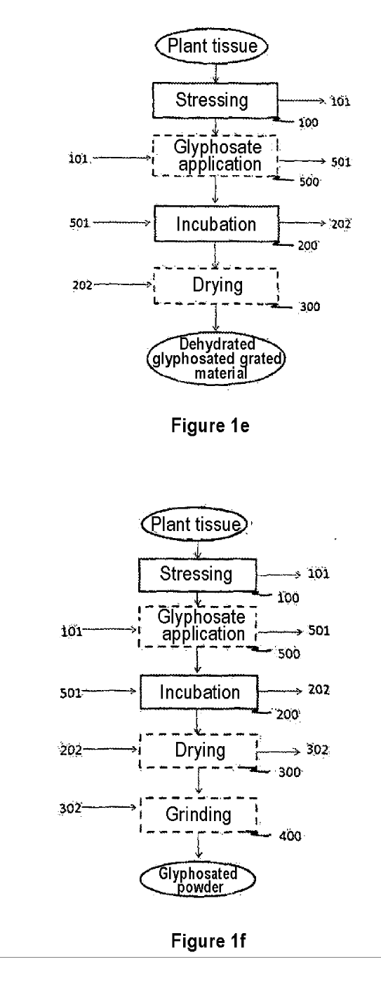

In general the process for the overproduction of SA and PC in horticultural crops is illustrated in FIG. 1, and includes the following stages:
a) Subjecting the horticultural crop which has been previously washed and sanitized to post-harvest abiotic stress (100).
   During this stage the plant tissue of the horticultural crop is cut, preferably shredded to obtain fragments thereof, in which wounding the crop (101) activates the primary and secondary metabolism of the plant tissue to direct the flow of carbon towards the production of SA and PC.
b) Incubating (200) the wounded plant tissue (101).
   In this stage the wounded plant tissue (101) obtained in stage a) is incubated; incubation is carried out under specific conditions to encourage the production and accumulation of SA and PC. The product obtained is referred to as nutraceutical grated material.
   Optionally a stage of drying may be included after stage b), and this is illustrated in FIG. 1b.
c) Drying (300) the plant material (201) obtained in b).
   This stage is carried out in order to remove excess water, which helps to preserve the plant material with the nutraceuticals accumulated, inhibiting the proliferation of microorganisms and making it difficult for it to putrefy. Water is removed by evaporation in order to obtain dehydrated plant material with nutraceuticals accumulated.
   A stage of grinding, illustrated in FIG. 1c, may also be included after stage c).
d) Grinding (400) the plant material with nutraceuticals accumulated (301) obtained in stage c).
   During this stage the dehydrated plant material with nutraceuticals accumulated is reduced to powder with a preferred residence time of between 1 and 5 minutes, until a particle size of preferably between 0.05 mm and 0.35 mm is obtained, thus yielding a uniform dispersion of the solid material known as nutraceutical powder.

In order to provide overproduction of shikimic acid and phenolic compounds it is proposed in this patent application that a stage of glyphosate application, shown in FIG. 1d, should be optionally included between stages a) and b) described above.
aa) Applying glyphosate (500) to the wounded plant tissue (101) obtained in stage a). In this stage the wounded plant tissue is treated with a solution of glyphosate so as to obtain stressed grated material (501) in this way, and this is subsequently delivered to the previously described stage b) in order to finally obtain glyphosated treated material.
   Optionally a drying stage, as shown in FIG. 1e, may be included after stage b).
c) Drying (300) the glyphosated treated material (202) obtained in b).
   This stage is carried out in order to eliminate water, which helps to preserve the glyphosate treated material, inhibiting the proliferation of microorganisms and making putrefaction difficult. Water is removed by evaporation in order to obtain dehydrated glyphosate treated material.
   Additionally a grinding stage, represented in FIG. 1f, may optionally be included after stage c).
d) Grinding (400) the dehydrated glyphosate treated material (302) obtained in stage c).
   During this stage, the dehydrated glyphosated treated material is reduced to powder with a preferred residence time of between 1 and 5 min, until it reaches a particle size of preferably between 0.05 mm and 0.35 mm, thus achieving uniform dispersion of the solid material, which is known as glyphosate treated plant powder.
   The glyphosated treated material, the dehydrated glyphosate treated plant material and the glyphosate treated plant powder (202, 302, 402) contain SA and PC without purification, so each of these products individually undergo a subsequent stage of recovery, purification and cleaning (600) to obtain waste plant material (601) and pure SA and PC (FIG. 1g).

In particular the plant material with nutraceuticals accumulated obtained in accordance with the process illustrated in FIG. 1 has the following characteristics: rough appearance, dehydrated, orange color with slight discoloration, slight reduction in size, minimum diameter of 2 mm, pH 4-6, moisture content 15-95%, ash 4-7%, fats 0.5-0.7%, proteins 6.5-9.5%, carbohydrates 30-45%, vitamins and minerals 15-250, 100-500 mg SA/kg dry base (ppm) and 1000-3000 mg equivalents of chlorogenic acid/kg dry base (ppm of PC). It is recommended that the plant material with nutraceuticals accumulated be stored under freezing conditions, preferably at 20° C. below zero, in conditions of total darkness. Its industrial application is as a raw material for the extraction of nutraceutical compounds with applications in the dietary supplements and food industry as an additive for the production of processed foods deriving from fruits and vegetables (soups, sauces, among others).

Specifically the dehydrated plant material with nutraceuticals accumulated obtained in accordance with the process described above and represented in FIG. 1b has the following characteristics: rough appearance, dehydrated, orange color, reduced size, minimum diameter of 2 mm, pH 4-6, moisture content 2-15%, water activity (25° C.) 0.3-0.4, ash 7-8.5%, fats 0.7-0.8%, proteins 9.5-11%, carbohydrates 45-50%, vitamins and minerals 25-30%, 100-500 mg SA/kg dry base (ppm) and 1000-3000 mg equivalents of chlorogenic acid/kg dry base (ppm of PC). The recommended storage conditions for the plant material with nutraceuticals accumulated are refrigeration at 20° C. below zero, in conditions of total darkness, before deciding upon the final destination of the industrial application. Industrial application is as a raw material for the extraction of nutraceutical compounds having applications in the dietary supplements industry and as a supplement for direct human consumption, and dehydrated food as an additive for the production of processed foods deriving from fruits and vegetables (soups, sauces, among others).

Characteristically the nutraceutical powder obtained in accordance with the process described above and represented in FIG. 1c has the following characteristics: orange color, particle size range between 23 and 30% retained on a sieve for particles larger than 0.29 mm, between 10 and 23% retained between 0.10 and 0.29 mm, and between 5 and 10% retained on a sieve for particles smaller than 0.10 mm, pH 4-6, moisture content 2-15%, water activity (25° C.) 0.3-0.4, ash 7-8.5%, fats 0.7-0.8%, proteins 9.5-11%, carbohydrates 45-50%, vitamins and minerals 25-30%, 100-500 mg SA/kg dry base (ppm) and 1000-3000 mg equivalents of chlorogenic acid/kg dry base (ppm of PC). It is recommended that the nutraceutical powder should be stored at a temperature of 20 to 25° C., in total darkness. Its industrial application is as a raw material for the extraction of nutraceutical compounds with applications in the dietary supplements industry and as an additive for applications in the meat products and processed vegetables industries.

In particular the glyphosate treated material obtained in accordance with the process which includes stages a), a′) and b) described previously and represented in FIG. 1d has the following characteristics: rough appearance, slight dehydration, orange color with slight discoloration, reduced size, minimum diameter of 2 mm, pH 3-7, moisture content 15-95%, ash 0.2-6%, fats 0.2-0.5%, proteins 0.2-0.5%, carbohydrates 0.5-30%, vitamins and minerals 0.3-15%, 2000-6000 mg SA/kg dry base (ppm) and 1000-3000 mg equivalents of chlorogenic acid/kg dry base (ppm of PC). Its industrial application is as a raw material for the extraction of nutraceutical compounds (phenolic compounds) with applications in the dietary supplements industry and shikimic acid with application in the pharmaceutical industry for the production of Tamiflu®.

In the case of the dehydrated glyphosate treated material obtained in accordance with stages a), a′) b) and c) described above and represented in FIG. 1e, this has the following characteristics: rough appearance, dehydration, orange color, reduced size, minimum diameter of 2 mm, pH 3-7, moisture content 2-15%, water activity (25° C.) 0.3-0.4, ash 6-10%, fats 0.7-0.8%, proteins 9.5-11%, carbohydrates 45-50%, vitamins and minerals 25-30%, 2000-6000 mg SA/kg dry base (ppm) and 1000-3000 mg equivalents of chlorogenic acid/kg dry base (ppm of PC). Its industrial application is as a raw material for the extraction of nutraceutical compounds (phenolic compounds) with applications in the dietary supplements industry and shikimic acid with application in the pharmaceutical industry for the production of Tamiflu®.

The glyphosate treated plant powder obtained in accordance with stages a), a′) b), c) and d) described above and represented in FIG. 1f has the following characteristics: orange color, particle size range between 23 and 30% retained on a sieve for particles larger than 0.29 mm, between 10 and 23% retained between 0.10 and 0.29 mm and between 5 and 10% retained on a sieve for particles smaller than 0.10 mm, pH 3-7, moisture content 2-15%, water activity (25° C.) 0.3-0.4, ash 6-10%, fats 0.7-0.8%, proteins 9.5-11%, carbohydrates 45-50% vitamins and minerals 25-30%, 2000-6000 mg SA/kg dry base (ppm) and 1000-3000 mg equivalents of chlorogenic acid/kg dry base (ppm of PC). Its industrial application is as a raw material for the extraction of nutraceutical compounds (phenolic compounds) having applications in the dietary supplements industry and shikimic acid having application in the pharmaceutical industry for the production of Tamiflu®.

Each of the stages mentioned above is described in detail below.

### —Preparation of the Horticultural Crop

The horticultural crop used in this example comprises carrots at any stage of maturity, including physiological and commercial maturity, and also all those which have been discarded for human consumption because they have factors which diminish their quality, such as wounding, bruising or any mechanical damage, which need to be washed and sanitized, usually using standard protocols with chlorinated water, although any other standard disinfection process for plant material may be used. The volume of water required will depend upon the quantity of plant material in the horticultural crop. This quantity is usually in a ratio of 1:2 to 1:4 (kg of plant tissue:liters of chlorinated water) so that said plant material is wholly submerged in the chlorinated water. The chlorine concentration will be from 150 to 250 ppm at a pH of between 6 and 7. The contact time between water and the plant material is usually 5 to 30 minutes depending upon the quantity of impurities present on the surface of the plant. The purpose of washing and disinfection is primarily to remove soil and fertilizer residues, bacteria and insects from the surface. Plant materials are capable of being contaminated with pathogens which are responsible for causing diseases. Likewise organisms should be removed to prevent them from growing on the plant tissue during the incubation stage in this way. It is essential that organisms be removed given that stressed plant materials may give rise to different responses during the process of the production of bioactive compounds because of the fact that their growth represents a biotic stress not considered. In addition to this, the removal of solid residues and organisms from the surface of the plant tissue assists the subsequent recovery and purification process in which the compounds that are of interest because of their application are isolated. Other additional operations which may form part of this stage of preparing the plant tissue may be directed towards the sorting and fractionation of different tissues (removal of thorns, seeds, etc.), the removal of extraneous solid material which cannot be removed as a result of the washing process, as well as any other type of operation which is appropriate to help subsequent stages in the process being suitably performed, depending upon the nature and origin of the plant tissue used.

Stage a)—Subjecting the Plant Tissue of the Horticultural Crop to Post-Harvest Abiotic Stress

After the carrots have been prepared they are subjected to post-harvest abiotic stress. The selected abiotic stress is wounding stress as this is a typical operation in the processing of plant material, being scalable and economical. However other post-harvest abiotic stresses, such as UV radiation, hyperoxia, application of plant hormones, controlled and modified atmospheres, gassing with ethylene (C₂H₄) for ripening, changes in storage temperature and desiccation may be used jointly with wounding stress to stimulate the response of the tissue even more.

The main purpose of grating is to induce wounding stress activating the primary metabolism of the plant, from which the carbon sources necessary for the biosynthesis of bioactive compounds are synthesized.

In order to induce primary metabolism in the plant tissue through the effect of wounding stress the plant tissue is wounded to a size of between 2 mm and 17 mm in diameter. This diameter coincides with the average diameter of commercial food processors. As already mentioned, this stage can be easily scaled up to an industrial level, as there are commercial food processors which can produce wounded plant material from 2 to 17 mm in diameter. Once the wounded material has been obtained, it is placed in open containers to encourage cell respiration in the horticultural crop in such a way that aerobic metabolism is induced, and this directs the flow of carbon to the production of the bioactive compounds of particular interest in this invention. An intermediate product known as wounded carrot is obtained during this stage.

Stage b)—Incubating the Wounded Carrot Under Propitious Conditions for the Production and Accumulation of the Compounds of Interest

The wounded carrot obtained in stage a) is incubated under conditions propitious for the overproduction and accumulation of the compounds of interest to occur. The propitious temperature and incubation time conditions for increasing the concentration of phenolic compounds are known in the state of the art [Jacobo-Velázquez, D. A., Martínez-Hernández, G. B., Rodríguez, S., Cao, C.-M., Cisneros-Zevallos, L. Plants as biofactories: Physiological role of reactive oxygen species on the accumulation of phenolic antioxidants in carrot tissue under wounding and hyperoxia stress. J. Agric. Food Chem. 2011, 59, 6583-6593], but merely reproducing incubation at the temperature reported in this stage is not sufficient; it has to be combined with other incubation parameters comprising a different incubation time, which has been disclosed by the inventors of this application during the period required by the Industrial Property Law as prior disclosure [Becerra-Moreno, A., Benavides, J., Cisneros-Zevallos, L., Jacobo-Velázquez, D. A. Plants as biofactories: glyphosate-induced production of Shikimic acid and phenolic antioxidants in wounded carrot tissue J. Agric. Food Chem. 2012, 60, 11378-11386] in such a way that during this stage the incubation temperature range is from 22 to 28° C., between 12 and 36 hours' storage, with a wide pressure range (10,132-1,013,250 Pa), with the use of atmospheric pressure (˜101,325 Pa) being preferred for practicality, with a relative humidity of 50 to 80%, in the presence or absence of light. Other incubation process parameters include, but are not restricted to, the composition of the atmosphere to which the tissue is exposed (particularly when wounding stresses and modified atmospheres are used in combination), and the nature and intensity of the light/radiation to which the mentioned tissue of the horticultural crop is exposed.

Once this stage has been completed a product known as plant material with nutraceuticals accumulated is obtained, and this has distinctive and unique characteristics which provide it with commercial value, as it is an excellent raw material for the extraction of nutraceutical compounds having applications in the dietary supplements and food industries as an additive for the production of processed foods deriving from fruits and vegetables (soups, sauces, among others).

Optionally, after stage b) there may be included a stage of: —Drying of the plant material with nutraceuticals accumulated. This stage is carried out in order to eliminate excess water, which helps to preserve the plant material with nutraceuticals accumulated inhibiting the proliferation of microorganisms and making putrefaction difficult.

Water is eliminated by evaporation in order to obtain dehydrated plant material with nutraceuticals accumulated. Different types of industrial equipment may be used to dry the plant material with nutraceuticals accumulated, natural or forced convection tunnel stoves, or natural or forced convection tray stoves being preferred. The former allows continuous flows, processing is relatively quick (particularly if the convection is forced) and significant quantities of material can be processed, the preferred residence time being between 1 and 60 minutes, the difference in moisture content between the tissue entering and leaving is between 70% and 2%, the temperature of the inlet air preferably not exceeding 120° C., the moisture content of the inlet air being between 0% and 50% humidity with an air flow of 0.05 to 300 m³ of air/min. In order to accelerate this stage it is preferable that forced convection equipment should be used. Once this stage has been completed a product known as dehydrated plant material with nutraceuticals accumulated is obtained, and its principal industrial application lies as a raw material for the extraction of nutraceutical compounds as dietary supplement additives and as a supplement for direct human consumption and dehydrated food as an additive for the production of processed foods derived from fruits and vegetables (soups, sauces, to mention some).

Additionally, after stage c), there is included a stage of:

—Grinding the dehydrated plant material with nutraceuticals accumulated. During this stage the dehydrated plant material with nutraceuticals accumulated is comminute to reduce it to very small particles in order to obtain a uniform dispersion of the solid material, thus obtaining a nutraceutical powder. The reduction in size is brought about by dividing or fractionating the sample by mechanical means until the desired size is achieved. The methods of reduction mostly used in grinding machines are: compression, impact, shear and wounding friction, in which intermediate and fine grinders (hammer grinders and vertical roller grinders) are preferred. The latter comprise two or more parallel steel rollers which rotating concentrically cause the dehydrated plant material with nutraceuticals accumulated to pass through the space between them. The main force applied is that of compression, with a preferred residence time of between 1 and 5 min until a particle size of preferably 0.05 mm to 0.35 mm is obtained. Once this stage has been completed a product known as nutraceutical powder is obtained, and its commercial value lies in its being an excellent raw material for the extraction of nutraceutical compounds having applications in the dietary supplements industry and as an additive with applications in the meat products and processed vegetables industries.

In order to potentiate the overexpression of SA and PC between stage a) of post-harvest abiotic stress and incubation stage b) the grated and stressed plant tissue may undergo a stage of: —Application of glyphosate to the stressed horticultural crop. During this stage the wounded plant tissue is treated with a solution of glyphosate through the submergence or spraying method. Application by spraying is preferred because it generates greater yields.

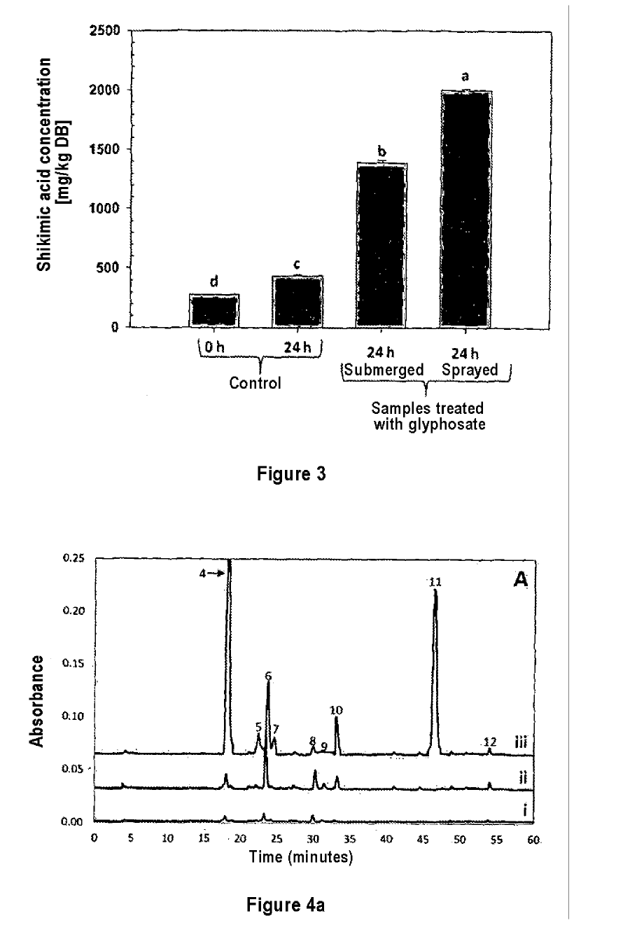

It has been found that spraying results in ≈44% more SA accumulation than in submerged samples, as shown in FIG. 3. This difference is attributed to the removal of signal molecules which induce the response to the wounding as part of the submergence process. The (at least partial) removal of extracellular adenosine triphosphate (eATP, produced by the wounding stress effect) occurs in the submerged material, and this may be related to lesser generation of reactive oxygen species (ROS), thus reducing the yield of SA in comparison with application by spraying. Abiotic wounding stress activates the metabolic pathways involved in the production of phenolic compounds while the application of glyphosate makes it possible to block the flow of SA to the production of other compounds, thus bringing about an accumulation of SA in the horticultural crop. Thus the wounded carrots have been treated with a solution of glyphosate (0-482 g/L) using one of the two different methods of application (submergence and spraying), the spraying method being preferred because of its better yields, and stored for 24 hours to determine the combined effect of wounding stress and glyphosate application on the accumulation of SA and PC. In the first (submerged) method of application the grated material was submerged in a ratio of 1:2 to 1:4 (kg of plant material:liters of glyphosate solution), in such a way that said material was wholly submerged in the glyphosate solution. Subsequently it was drained for approximately 10-30 min. In the second method of application (spraying) the grated material was sprayed in a ratio of 1:⅙ to 1:½ (kg of plant material:liters of glyphosate solution), using a commercial sprayer. It is of particular interest for this invention that the glyphosate solutions which have to be applied should be prepared as free from harm as possible so as not to encourage the growth of microorganisms. As mentioned previously, plant materials are likely to be contaminated with pathogens responsible for causing diseases, above all during the stage of incubating the stressed horticultural crop, giving rise to metabolic responses other than production of the bioactive compounds of interest. Thus these glyphosate solutions should be prepared using twice-distilled water (td H₂O) with a pH of between 6 and 7. The effect of the pH of the water on the effectiveness of commercial formulations of glyphosate herbicide has been investigated and it has been shown that the pH of the water used (1.5-6) does not affect the control of diseases in any of the commercial formulations, as described by Penner, D. and Michael, J. in the article entitled, “The effect of pH on glyphosate activity and water conditioning” in ASTM Int. 2010, 7, 1-6. This is of vital importance for this invention, given that it guarantees that the effectiveness of the glyphosate (0-482 g/L) used will be kept stable using twice-distilled water with a pH of between 6 and 7.

As part of this research it has been demonstrated that for the particular case of carrot tissue, it is possible to obtain a significant concentration of SA without the need to apply glyphosate, something which has not been reported by any other author, so application of the glyphosate application stage to the stressed horticultural crop may be optional if the aim of the process is to obtain SA from carrot tissue. This makes it possible to reduce the costs of the process by eliminating a stage and avoiding the use of reagents (glyphosate). However the yield of SA per mass of horticultural crop is lower than when glyphosate is applied, see FIG. 3, which shows the concentration of shikimic acid in control samples, that is to say without the application of glyphosate and with the application of sprayed or submerged glyphosate.

Optionally, after the stages in the sequence of stages a), a′), and b) there may be included a stage of: —Drying of the glyphosated treated material, which is carried out as described previously in stage c) of drying of the plant material with nutraceuticals accumulated, except that in this case this stage is carried out on glyphosate treated material. Once this stage has been concluded a product known as dehydrated glyphosate treated material is obtained, and the principal industrial application of this lies in its being a raw material for the extraction of nutraceutical compounds (phenolic compounds) with applications in the dietary supplements industry and shikimic acid with application in the pharmaceutical industry for the production of Tamiflu. Additionally, after the sequence of stages a), a′), b) and c), there is optionally included a stage of: —Grinding of the dehydrated glyphosate treated material, which is carried out as described previously in stage d). After this stage has been completed a product known as glyphosate powder is obtained, and its main industrial application lies in being, like the dehydrated glyphosated grated material, a raw material for the extraction of nutraceutical compounds (phenolic compounds) with applications in the dietary supplements industry, and shikimic acid with application in the pharmaceutical industry for the production of Tamiflu®.

### PREFERRED EMBODIMENTS

### Example 1

### Effect of Wounding Stress on the Accumulation of SA

The plant material used in this example comprised carrots (Daucus carota) obtained from a local supermarket (Monterrey, N. L., Mexico), which were washed and disinfected with chlorinated water in a concentration of 200 ppm, pH 6.5, in a ratio of 1:3 kg of plant material:liters of chlorinated water, for 10 min.

Stage a) The previously washed and disinfected carrots were subjected to post-harvest abiotic stress.

This stage was carried out by grating the carrot with a commercial vegetable grater. Stressed grated carrots having a diameter of 0.7 cm were obtained.

Stage b) Incubation of the grated carrot stressed in a) to encourage the production and accumulation of SA and PC.

In particular 300 grams of stressed grated carrot obtained in stage a) were placed in open plastic containers having a capacity of 5.7 L (Sterilite, Townsend, USA) and incubated in an incubator for 48 hours (VWR, Radnor, USA) at 25° C. and at atmospheric pressure with a relative humidity of 65%, in total darkness, and samples were collected every 24 hours to determine the time at which the maximum accumulation of SA occurred.

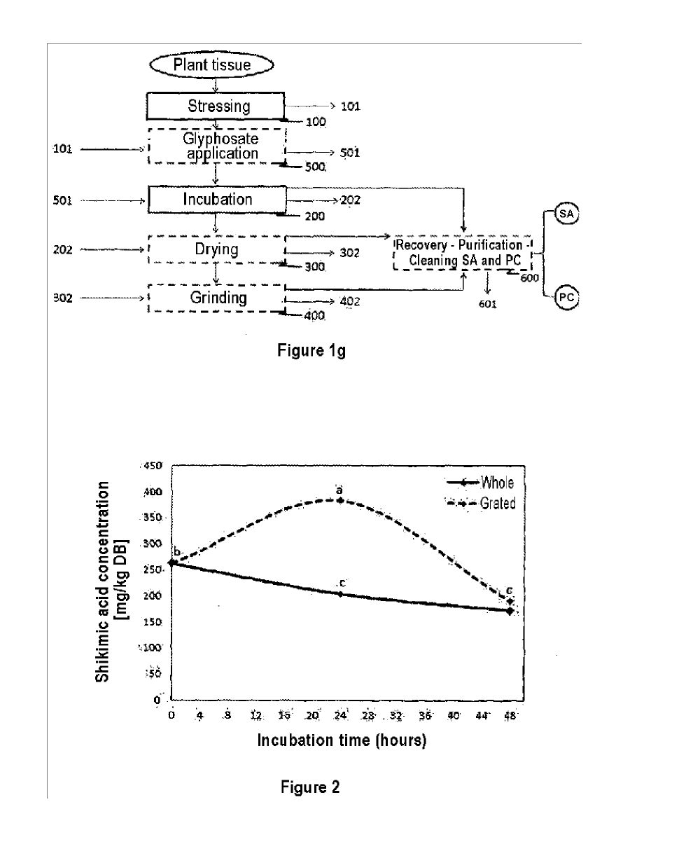

The SA concentration in the nutraceutical grated material increased during the first hours of storage, the maximum concentration being observed at 24 hours (FIG. 2). The concentration of SA in grated carrot stored for 24 hours increased by ˜50% in comparison with the grated carrot prior to storage (0 hours). This demonstrates that it is possible to produce SA from carrot tissue which has been stressed by wounding without the need to carry out the stage of applying glyphosate to the stressed horticultural crop, although the concentration of SA obtained is less than when glyphosate is applied to the stressed tissue (Table 2).

There are no reports in the literature which describe the effect of applying abiotic stresses on the accumulation of SA in plant tissues. When carrots are exposed to wounding stress, proteins and secondary metabolites such as phenolic compounds are synthesized during the process of acclimatization, SA being a primary precursor metabolite for the synthesis of aromatic amino acids, which in turn are used for the synthesis of said proteins and secondary metabolites such as phenolic compounds. Thus it is likely that the accumulation of SA after 24 hours of storage is related to a higher rate of synthesis of this compound in comparison with its rate of use in the synthesis of amino acids and secondary metabolites.

TABLE 2
Difference in the concentrations of SA and PC
in nutraceutical grated material produced by stress,
incubation and without the application of glyphosate
and glyphosated grated material produced by stress,
incubation and glyphosate.

| | | PC |
|---|---|---|
| | SA | mg equivalents of |
| | mg SA/kg dry | chlorogenic acid/kg |
| Product | base (ppm) | dry base (ppm) |
| Nutraceutical | 100-500 | 1000-3000 |
| grated material | | |
| Glyphosated | 2000-6000 | 1000-3000 |
| grated material | | |

### Example 2

### Effect of Drying on the Accumulation of SA

The plant material used in this example comprised carrots (Daucus carota) obtained from a local supermarket (Monterrey, N. L., Mexico), which were washed and disinfected with chlorinated water in a concentration of 200 ppm, pH 6.5, in a ratio of 1:3 kg of plant material:liters of chlorinated water, for 10 min.

Stage a) the Previously Washed and Disinfected Carrots were Subjected to Post-Harvest Abiotic Stress.

This stage was carried out by grating the carrot with a commercial vegetable grater. Stressed grated carrots having a diameter of 0.7 cm were obtained.

Stage b) Incubation of the Grated Carrot Stressed in a) to Encourage the Production and Accumulation of SA and PC.

In particular 300 grams of stressed grated carrot obtained in stage a) were placed in open plastic containers having a capacity of 5.7 L (Sterilite, Townsend, USA) and incubated in an incubator for 48 hours (VWR, Radnor, USA) at 25° C. and at atmospheric pressure with a relative humidity of 65%, in total darkness, and samples were collected every 24 hours to determine the time at which the maximum accumulation of SA occurred.

### Stage c) Drying of the Nutraceutical Grated Material Obtained in b) to Obtain Dehydrated Nutraceutical Grated Material.

This stage was carried out by placing the nutraceutical grated material in a forced convection tunnel stove for a residence time of 60±2 minutes, with an inlet air temperature of 80±5° C., a moisture content of the inlet air between 15±5% humidity and an air flow of 200 m³ of air/min.

The concentration of SA increased during the first hours of storage, the maximum concentration being observed at 24 hours. The SA concentration in grated carrot stored for 24 hours increased ˜45% in comparison with the grated carrot prior to storage (0 hours). The SA concentration in the dehydrated nutraceutical grated material stored for 24 hours increased ˜40% with respect to the grated carrot prior to storage (0 hours).

### Example 3

### Effect of Grinding on the Accumulation of SA

The plant material used in this example comprised carrots (Daucus carota) obtained from a local supermarket (Monterrey, N. L., Mexico), which were washed and disinfected with chlorinated water in a concentration of 200 ppm, pH 6.5, in a ratio of 1:3 kg of plant material:liters of chlorinated water, for 10 min.

Stage a) the Previously Washed and Disinfected Carrots were Subjected to Post-Harvest Abiotic Stress.

This stage was carried out by grating the carrot with a commercial vegetable grater. Stressed grated carrots having a diameter of 0.7 cm were obtained.

### Stage b) Incubation of the Grated Carrot Stressed in a) to Encourage the Production and Accumulation of SA.

In particular 300 grams of stressed grated carrot obtained in stage a) were placed in open plastic containers having a capacity of 5.7 L (Sterilite, Townsend, USA) and incubated in an incubator for 48 hours (VWR, Radnor, USA) at 25° C. and at atmospheric pressure with a relative humidity of 65%, in total darkness, and samples were collected every 24 hours to determine the time at which the maximum accumulation of SA occurred.

### Stage c) Drying of the Nutraceutical Grated Material Obtained in b) to Obtain Dehydrated Nutraceutical Grated Material.

This stage was carried out by placing the nutraceutical grated material in a forced convection tunnel stove for a residence time of 60±2 minutes, with an inlet air temperature of 80±5° C., a moisture content of the inlet air between 15±5% humidity and an air flow of 200 m³ of air/min.

### Stage d) Grinding of the Dehydrated Nutraceutical Grated Material Obtained in c) to Obtain a Nutraceutical Powder.

In this stage the dehydrated nutraceutical grated material was comminuted in a vertical roller mill preferably with two steel rollers parallel to each other rotating concentrically, pushing the dehydrated nutraceutical grated material through the space between them, with a residence time of 3 minutes±1, and a particle size of 0.20 mm±0.05.

The SA concentration increased during the first hours of storage, the maximum concentration being observed at 24 hours. The SA concentration in grated carrot stored for 24 hours increased ˜48% in comparison with the grated carrot prior to storage (0 hours).

The concentration of SA in the nutraceutical powder stored for 24 hours increased ˜37% in comparison with the grated carrot prior to storage (0 hours).

This shows that it is possible to produce SA from wounding-stressed carrot tissue, which is subsequently subjected to a drying process and finally ground, without the need to carry out the stage of applying glyphosate to the stressed horticultural crop, even when the concentration of SA obtained is less than when glyphosate is applied to the stressed tissue (see FIG. 3).

### Example 4

### Effect of the Method of Application (Submergence or Spraying) of a Glyphosate Solution (482 g/L) on the Overproduction and Accumulation of SA on Grated Horticultural Crops after 24 Hours of Storage

The plant material used in this example comprised carrots (Daucus carota) obtained from a local supermarket (Monterrey, N. L., Mexico), which were washed and disinfected with chlorinated water in a concentration of 200 ppm, pH 6.5, in a ratio of 1:3 kg of plant material:liters of chlorinated water, for 10 min.

Stage a) the Carrots were Subjected to Post-Harvest Abiotic Stress.

This stage was carried out by grating the carrot with a commercial vegetable grater. Stressed grated carrots having a diameter of 0.7 cm were obtained.

Stage a′) Application of Glyphosate to the Horticultural Crop Stressed in a).

In this stage, the stressed grated plant tissue was treated with a concentrated solution of glyphosate (482 g/L) using two different methods of application (submergence and spraying). In particular 300 grams of stressed grated carrot obtained in stage a) were placed in open plastic containers having a capacity of 5.7 L (Sterilite, Townsend, USA). In the first method of application (submergence) the grated material was submerged in 500 mL of the glyphosate solution for 2 min and subsequently drained for 10 min. In the second method of application (spraying) 100 mL of the glyphosate solution was sprayed on the grated carrot using a commercial sprayer.

Stage b) Incubation of the Grated Carrot Stressed in a′) to Encourage the Overproduction and Accumulation of SA.

Finally, the treated grated material was incubated at 25° C. for 24 hours under atmospheric pressure with a relative humidity of 65%, in total darkness, to determine the combined effect of wounding stress and glyphosate application on the accumulation of SA.

The grated carrot sprayed with glyphosate showed an increase of ˜44% in SA accumulation in comparison with the submerged samples (FIG. 3).

### Example 5

### Effect of the Concentration of Sprayed Glyphosate (in a Concentration Equal to or Less than 482 g/L) on the Overproduction and Accumulation of SA and PC in Grated Horticultural Crop after 24 Hours of Storage

The plant material used in this example comprised carrots (Daucus carota) obtained from a local supermarket (Monterrey, N. L., Mexico), which were washed and disinfected with chlorinated water in a concentration of 200 ppm, pH 6.5, in a ratio of 1:3 kg of plant material:liters of chlorinated water, for 10 min.

Stage a) the Carrots were Subjected to Post-Harvest Abiotic Stress.

This stage was carried out by grating the carrot with a commercial vegetable grater. Stressed grated carrots having a diameter of 0.7 cm were obtained.

Stage a′) Application of Glyphosate to the Horticultural Crop Stressed in a).

In this stage, the stressed grated plant tissue was treated by spraying 100 mL of glyphosate solution at different concentrations using a commercial sprayer.

Here the glyphosate solution with which the grated material was separately treated had a concentration of 100, 200, 300, 400 and 482 g/L; and a control sample on which 100 ml of water without glyphosate was sprayed, this being referred to as glyphosate concentration 0.

Stage b) Incubation of the grated carrot stressed in a′) to encourage the overproduction and accumulation of SA. Finally, the treated grated material was incubated at 25° C. for 24 hours at atmospheric pressure with a relative humidity of 65%, in total darkness, to determine the combined effect of wounding stress and glyphosate application at different concentrations on the accumulation of SA and PC.

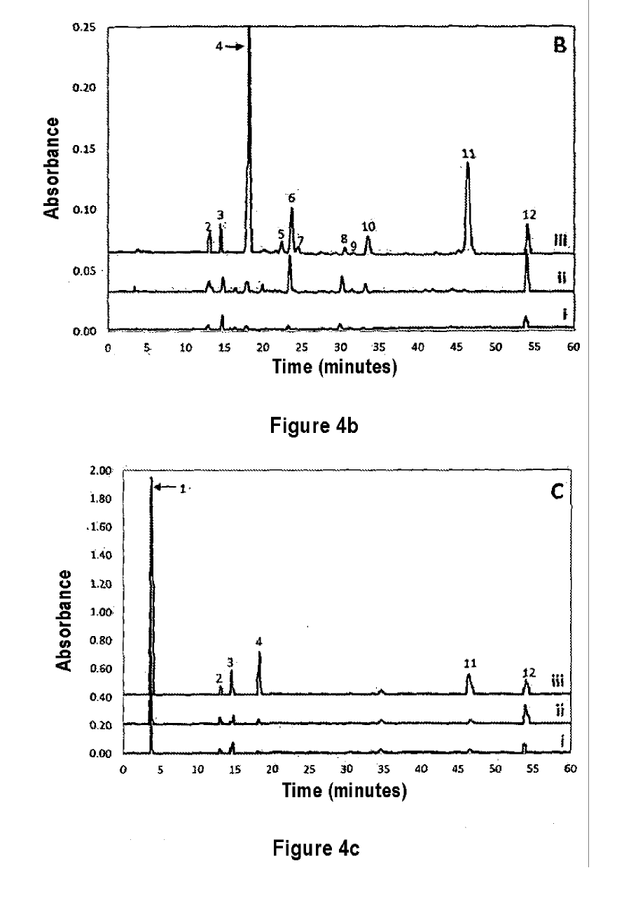

The SA and PC content in grated carrot samples which were and were not sprayed with solutions containing different concentrations of glyphosate (0, 100, 200, 300, 400 and 482 g/L) was determined by HPLC-PDA. FIGS. 4a, 4b and 4c and Table 3 show the identity of the accumulated compounds (SA and various PC) as part of the process of wounding stress and glyphosate application to carrot tissue, while Table 4 shows the quantities of these compounds when different concentrations of glyphosate were used.

TABLE 3
Tentative identification of shikimic acid and phenolic
compounds in carrot tissue obtained by HPLC-PDA.

| Peak number^{a} | | | | |
|---|---|---|---|---|
| (retention | | Tentative | Previously reported in | Method of |
| time) | λmax^{b} (nm) | identification | carrot^{c} | identification^{d} |
| 1 (3.7) | 215 | SA | | A, B |
| 2 (13.0) | 215, 253, 290 | PA | iv | A, B, C |
| 3 (14.8) | 217, 271 | GAD | | A |
| 4 (18.1) | 217, 242, 320 | CA | i, ii, iii, iv, v, | A, B, C |
| | | | vi | |
| 5 (22.2) | 217, 238, 325 | 3,5-diCQA | ii, iii, iv, v, vi | A, B, C |
| 6 (23.2) | 228, 313 | pCADA | | A |
| 7 (24.1) | 215, 240, 326 | 4,5 diCQA | i, ii, iii, iv, v, | A, B, C |
| | | | vi | |
| 8 (29.9) | 226, 310 | pCA | i | A, B, C |
| 9 (31.4) | 217, 237, 323 | FA | i, iii, iv, v, vi | A, B |
| 10 (33.2) | 225, 313 | pCADB | | A |
| 11 (45.8) | 218, 239, 328 | FAD | | A, B |
| 12 (53.9) | 215, 268, 301 | IC | iii, iv, vi | B, C |

^{a}Peak number assigned in accordance with the order of elution on the C18 stationary phase (FIG. 4).
^{b}Maximum absorption wavelengths in the UV/Vis spectrum for each chromatographic peak.
^{c}Previously reported.
^{d}Method used for tentative peak identification: (A) Identification by comparing the retention time and maximum absorption wavelengths in the UV/Vis spectrum of commercial standards; (B) Identification by spectral interpretation of maximum absorption wavelengths in the UV/Vis spectrum and comparison with maximum absorption wavelengths in the literature; (C) Identification by chromatographic elution order reported in the literature.
Abbreviations:
shikimic acid dicaffeoylquinic acid (SA),
protocatechuic acid (PA),
gallic acid derivative (GAD);
chlorogenic acid (CA),
3,5-dicaffeoylquinic acid (3,5-diCQA),
p-coumaric acid derivative A (pCADA),
4,5-dicaffeoylquinic acid (4,5-diCQA),
p-coumaric acid (pCA),
ferulic acid (FA),
p-coumaric acid derivative B (pCADB),
ferulic acid derivative (FAD),
isocoumarin (IC).

TABLE 4
Behavior of the concentrations of shikimic acid and individual phenolic compounds in the
methanol extracts of grated carrots exposed to wounding stress, with and without spraying with
solutions of glyphosate at different concentrations, and stored at 25° C. for 24 hours.

| | | Concentrations of shikimic acid and individual phenolic compounds | | | | | | | |
|---|---|---|---|---|---|---|---|---|---|
| | | (mg/kg DB) ^{a, b, c} | | | | | | | |
| | | | Grated samples stored at 25° C. for 24 hours | | | | | | |
| Peak | | Control | | Glyphosate concentrations in the sprayed solution (g/L) | | | | | |
| number | Compound | t 0 hrs | Control | 0 | 100 | 200 | 300 | 400 | 482 |
| 1 | SA | 259.1 ± | 458.8 ± | 323.1 ± | 789.8 ± | 1250.9 ± | 2538.3 ± | 4306.3 ± | 4755.3 ± |
| | | 4.7 g | 6.3 f | 7.1 fg | 62.3 c | 72.0 d | 56.9 c | 79.1 b | 117.9 a |
| 2 | PA | 111.2 ± | 206.5 ± | 85.7 ± | 58.0 ± | 40.1 ± | 25.3 ± | 45.8 ± | 58.8 ± |
| | | 1.1 b | 10.1 a | 4.0 c | 2.6 de | 3.1 f | 0.7 g | 1.8 cf | 1.1 d |
| 3 | GAD | 286.5 ± | 205.1 ± | 167.2 ± | 198.4 ± | 196.2 ± | 220.4 ± | 229.4 ± | 254.1 ± |
| | | 4.8 a | 1.4 c | 3.3 e | 1.6 c | 2.1 c | 7.9 c | 8.4 c | 8.9 a |
| 4 | CA | 18.4 ± | 50.1 ± | 46.1 ± | 21.9 ± | 147.5 ± | 346.7 ± | 745.0 ± | 1044.2 ± |
| | | 0.3 e | 4.3 e | 3.4 e | 2.8 e | 5.6 d | 11.8 c | 27.1 b | 29.0 a |
| 5 | 3,5-diCQA | ND | ND | ND | 2.8 ± | 13.0 ± | 14.9 ± | 17.8 ± | 29.4 ± |
| | | | | | 0.0 d | 0.2 c | 0.4 bc | 0.6 b | 3.8 a |
| 6 | pCADA | 24.5 ± | 204.2 ± | 212.4 ± | 25.3 ± | 38.6 ± | 50.1 ± | 68.4 ± | 195.3 ± |
| | | 1.2 d | 13.5 a | 19.6 a | 1.0 d | 0.9 cd | 1.2 bc | 2.6 b | 11.8 a |
| 7 | 4,5 diCQA | ND | ND | ND | 3.0 ± | 6.0 ± | 6.7 ± | 10.0 ± | 14.8 ± |
| | | | | | 0.0 d | 0.0 c | 0.0 c | 0.2 b | 0.6 a |
| 8 | pCA | 27.3 ± | 78.6 ± | 46.4 ± | 36.7 ± | 32.1 ± | 19.9 ± | 11.4 ± | 5.7 ± |
| | | 0.7 e | 1.2 a | 1.6 b | 1.9 c | 1.2 d | 0.9 f | 0.4 g | 0.1 h |
| 9 | FA | 5.1 ± | 13.4 ± | 4.8 ± | 6.5 ± | 7.1 ± | 4.6 ± | 3.1 ± | 3.0 ± |
| | | 0.0 b | 0.9 a | 0.0 b | 0.0 c | 0.1 c | 0.0 c | 0.0 d | 0.0 d |
| 10 | pCADB | 7.9 ± | 52.1 ± | 4 7.4 ± | 14.2 ± | 43.7 ± | 64.4 ± | 90.7 ± | 135.8 ± |
| | | 0.3 f | 1.8 d | 1.6 de | 0.4 f | 2.2 E | 3.8 c | 4.5 b | 5.6 a |
| 11 | FAD | ND | ND | ND | 212.4 ± | 319.4 ± | 437.9 ± | 548.5 ± | 659.1 ± |
| | | | | | 7.5 e | 9.6 D | 13.2 c | 16.3 b | 15.7 a |
| 12 | IC | 36.8 ± | 178.4 ± | 126.8 ± | 22.4 ± | 17.9 ± | 16.9 ± | 27.6 ± | 70.6 ± |
| | | 0.8 D | 2.8 a | 1.3 b | 0.3 f | 0.2 g | 0.3 g | 0.6 e | 1.1 c |

(a) Concentrations are expressed as equivalents of chlorogenic acid for peaks 5, 7 and 12; as equivalents of gallic acid for peak 3; as equivalents of p-coumaric acid for peaks 6 and 10, and as equivalents of ferulic acid for peak 11.
(b) The compounds were quantified at 215 nm (peak 1), 280 nm (peaks 2, 3 and 12) and 320 nm (peaks 4, 5, 6, 7, 8, 9, 10 and 11).
(c) The values represent the average of 4 repetitions ± standard error in the mean.
(d) Different letters in the same row indicate statistically significant differences in the LSD test (p < 0.05).
ND =Not detected.
Abbreviations: shikimic acid (SA), protocatechuic acid (PA), gallic acid derivative (GAD); chlorogenic acid (CA), 3,5-dicaffeoylquinic acid (3,5-diCQA), p-coumaric acid derivative A (pCADA), 4,5-dicaffeoylquinic acid (4,5-diCQA), p-coumaric acid (pCA), ferulic acid (FA), p-coumaric acid derivative B (pCADB), ferulic acid derivative (FAD), isocoumarin (IC).

Based on the quantitative determinations using HPLC-PDA it was estimated that the increase in the concentration of SA in grated carrot which was not sprayed with glyphosate was ˜77% after 24 hours' storage. The maximum accumulation of SA was obtained when the grated carrot was sprayed with a solution of 482 g/L of glyphosate.

As for the PC, 3,5-dicaffeoylquinic acid (3,5-diCQA), 4,5-dicaffeoylquinic acid (4,5-diCQA) and a ferulic acid derivative (FAD) were only identified in the samples treated with glyphosate. Wounding stress induced the accumulation of protocatechuic acid (PA), CA, p-coumaric acid (pCA), pCA derivative A (pCADA), pCA derivative B (pCADB), ferulic acid (FA) and isocoumarin (IC). The PC showing the highest percentage increase in its concentration was pCADA, followed by pCADB, IC, pCA, FA and PA. The application of glyphosate to carrots stressed by wounding induced the accumulation of some hydroxycinnamic acids (CA, 3,5-diCQA, 4,5-diCQA, pCADBA and FAD) after 24 hours' storage in comparison with samples to which no glyphosate was applied, while the PA concentration remained constant. The PC showing the greatest increase in its concentration through the effect of applying glyphosate to grated carrot was CA. Additionally the application of glyphosate induced the synthesis and accumulation of 3,5-diCQA and 4,5-diCQA, although these compounds were not detected in the treatments in which only wounding stress was used.

This has been the first investigation to evaluate the effect of applying glyphosate to the accumulation of individual PC in wounding-stressed plant material. Previous reports indicate that the biosynthesis of PC and hydroxycinnamic acids in plants treated with glyphosate is inhibited due to the effect of that herbicide on the conversion of SA to L-phenylalanine. However in this investigation the application of glyphosate to wounding-stressed carrot gave rise to a significant accumulation of some hydroxycinnamic acids such as chlorogenic acid and its derivatives.

### Example 6

### Overproduction and Accumulation of Bioactive Compounds Through the Application of Abiotic Stress and Glyphosate to Carrot Tissue

The plant material used in this example comprised carrots (Daucus carota) obtained from a local supermarket (Monterrey, N. L., Mexico), which were washed and disinfected with chlorinated water in a concentration of 200 ppm, pH 6.5, in a ratio of 1:3 kg of plant material:liters of chlorinated water, for 10 min.

Stage a) the Carrots were Subjected to Post-Harvest Abiotic Stress.

This stage was carried out by grating the carrot with a commercial vegetable grater. Stressed grated carrots having a diameter of 0.7 cm were obtained.

Stage a′) Application of Glyphosate to the Horticultural Crop Stressed in a).

In this stage, the stressed grated plant tissue was treated by spraying (commercial sprayer) 100 mL of a concentrated solution of glyphosate (482 g/L).

Stage b) Incubation of the Grated Carrot Stressed in a′) to Encourage the Overproduction and Accumulation of SA.

Finally, the treated grated material was incubated at 25° C. for 24 hours at atmospheric pressure with a relative humidity of 65%, in total darkness, to determine the combined effect of wounding stress and glyphosate application on the accumulation of SA and PC.

The maximum accumulation of SA was obtained when the grated carrot was sprayed with a solution of 482 g/L of glyphosate. Under these conditions the SA concentration increased by some ˜1735% after 24 hours of storage in comparison with the control samples (0 hours).

The PC showing the greatest increase in concentration through the effect of applying glyphosate to grated carrot was chlorogenic acid (CA). The CA content in the treatment in which the 482 g/L of glyphosate solution was used showed an increase of ˜1980% in comparison with the control samples at 24 hours.

This demonstrates the overproduction of SA and PC when glyphosate is applied after wounding stress (Table 2).

TABLE 2
Difference in the concentration of SA and PC
in nutraceutical grated material produced by stress,
incubation and without the application of glyphosate
and glyphosated grated material produced by stress,
incubation and glyphosate.

| | | PC |
|---|---|---|
| | SA | mg equivalents of |
| | mg SA/kg dry | chlorogenic acid/kg |
| Product | base (ppm) | dry base (ppm) |
| Nutraceutical | 100-500 | 1000-3000 |
| grated material | | |
| Glyphosated | 2000-6000 | 1000-3000 |
| grated material | | |

### Example 7

### Effect of Drying on the Overproduction and Accumulation of Bioactive Compounds Through the Application of Abiotic Stress and Glyphosate to Carrot Tissue

The plant material used in this example comprised carrots (Daucus carota) obtained from a local supermarket (Monterrey, N. L., Mexico), which were washed and disinfected with chlorinated water in a concentration of 200 ppm, pH 6.5, in a ratio of 1:3 kg of plant material:liters of chlorinated water, for 10 min.

Stage a) the Carrots were Subjected to Post-Harvest Abiotic Stress.

This stage was carried out by grating the carrot with a commercial vegetable grater. Stressed grated carrots having a diameter of 0.7 cm were obtained.

Stage a′) Application of Glyphosate to the Horticultural Crop Stressed in a).

In this stage, the stressed grated plant tissue was treated by spraying (commercial sprayer) 100 mL of a concentrated solution of glyphosate (482 g/L).

Stage b) Incubation of the Grated Carrot Stressed in a′) to Encourage the Overproduction and Accumulation of SA.

The treated grated material was incubated at 25° C. for hours at atmospheric pressure with a relative humidity of 65%, in total darkness, to determine the combined effect of wounding stress and glyphosate application on the accumulation of SA and PC.

### Stage c) Drying of the Glyphosated Grated Material Obtained in b) to Produce Dehydrated Glyphosated Grated Material.

This stage was carried out by placing the glyphosated grated material in a forced convection tunnel stove with a residence time of 60±2 minutes, an inlet air temperature of 80±5° C., an inlet air humidity between 15±5% humidity and an air flow of 200 m³ of air/min.

The maximum SA accumulation was obtained when the grated carrot was sprayed with a solution of 482 g/L of glyphosate. Under these conditions the SA concentration increased by some ˜1766% after 24 hours' storage in comparison with the control samples (0 hours). The SA concentration increased some ˜1550% in the dehydrated glyphosated grated material stored for 24 hours in comparison with the grated carrot prior to storage (0 hours).

The PC showing the greatest increase in concentration through the effect of the application of glyphosate to grated carrot was chlorogenic acid (CA). The CA content in the treatment in which the 482 g/L of glyphosate solution was used showed an increase of ˜1930% in comparison with the control samples at 24 hours. The CA concentration increased by some ˜1700% in the dehydrated glyphosated grated material stored for 24 hours in comparison with the grated carrot before storage (0 hours).

### Example 8

### Effect of Grinding on the Overproduction and Accumulation of Bioactive Compounds Through the Application of Abiotic Stress and Glyphosate to Carrot Tissue

The plant material used in this example comprised carrots (Daucus carota) obtained from a local supermarket (Monterrey, N. L., Mexico), which were washed and disinfected with chlorinated water in a concentration of 200 ppm, pH 6.5, in a ratio of 1:3 kg of plant material:liters of chlorinated water, for 10 min.

Stage a) the Carrots were Subjected to Post-Harvest Abiotic Stress.

This stage was carried out by grating the carrot with a commercial vegetable grater. Stressed grated carrots having a diameter of 0.7 cm were obtained.

Stage a′) Application of Glyphosate to the Horticultural Crop Stressed in a).

In this stage, the stressed grated plant tissue was treated by spraying (commercial sprayer) 100 mL of a concentrated solution of glyphosate (482 g/L).

Stage b) Incubation of the Grated Carrot Stressed in a′) to Encourage the Overproduction and Accumulation of SA.

The treated grated material was incubated at 25° C. for hours at atmospheric pressure with a relative humidity of 65%, in total darkness, to determine the combined effect of wounding stress and glyphosate application on the accumulation of SA and PC.

### Stage c) Drying of the Glyphosated Grated Material Obtained in b) to Produce Dehydrated Glyphosated Grated Material.

This stage was carried out by placing the glyphosated grated material in a forced convection tunnel stove with a residence time of 60±2 minutes, an inlet air temperature of 80±5° C., an inlet air moisture content between 15±5% humidity and an air flow of 200 m³ of air/min.

Stage d) Grinding of the Dehydrated Glyphosated Grated Material Obtained in c) to Produce a Glyphosated Powder.

In this stage the dehydrated glyphosated grated material was comminuted in a vertical roller mill, preferably comprising two steel rollers parallel to each other rotating concentrically, pushing the dehydrated nutraceutical grated material through the space between them, with a residence time of 3±1 min. and a particle size of 0.20±0.05 mm.

The maximum accumulation of SA was obtained when the grated carrot was sprayed with a solution of 482 g/L of glyphosate. Under these conditions the SA concentration increased by some ˜1720% after 24 hours of storage in comparison with the control samples (0 hours). The SA concentration increased by some ˜1470% in the glyphosated powder stored for 24 hours in comparison with the grated carrot before storage (0 hours).

The PC showing the greatest increase in concentration through the effect of applying glyphosate to grated carrot was chlorogenic acid (CA). The CA content in the treatment in which the 482 g/L of glyphosate solution was used showed an increase of ˜1940% in comparison with the control samples at 24 hours. The CA concentration increased by some ˜11620% in the dehydrated glyphosated grated material stored for 24 hours in comparison with the grated carrot before storage (0 hours).

This demonstrates that it is possible to overproduce SA and PC from wounding-stress glyphosated carrot tissue which subsequently underwent a process of drying and finally grinding.

### Example 9

### Preparation of a Sample for the Analysis of Plant Chemicals

The SA was extracted from the horticultural crop using two different processes based on the method of detection and quantification which was to be used (spectrophotometry or chromatography). For spectrophotometric analysis of the SA the latter was extracted using the method reported by Zelaya, I. A., Anderson, J. A., Owen, M. D. and Landes, R. D.

Evaluation of spectrophotometric and HPLC methods for shikimic acid determination in plants: models in glyphosate-resistant and susceptible crops. J. Agric. Food Chem. 2011, 59, 2202-2212, with a few modifications. The carrot tissue (0.2 g) was homogenized with 0.25 N HCl (4 mL). Homogenization was carried out using a commercial cross-blade homogenizer, (Advanced Homogenizing System, VWR, Radnor, USA). Subsequently the homogenized material was stirred by inversion for 10 min in a Glas-Col stirrer (Terre Haute, Ind., USA) at 60 rpm and subsequently centrifuged (10 000×g, 15 min, 4° C.). The clarified supernatant (SA extract) was microfiltered using 0.45 μm nylon membranes (VWR, Radnor, USA).

For the identification and chromatographic quantification (HPLC-PDA) of SA and PC the carrot tissue (5 g) was homogenized with methanol (20 mL). The homogenized material was stored overnight (˜12 hours, 4° C.) and centrifuged (10 000×g, 15 min, 4° C.)

The clarified supernatant (methanolic extract) was microfiltered using 0.45 μm nylon membranes (VWR, Radnor, USA).

### Example 10

### Spectrophotometric Quantification of Shikimic Acid (SA)

The SA extracts were analyzed following a method reported by Zelaya, I. A., Anderson, J. A., Owen, M. D. and Landes, R. D. Evaluation of spectrophotometric and HPLC methods for shikimic acid determination in plants: models in glyphosate-resistant and susceptible crops. J. Agric. Food Chem. 2011, 59, 2202-2212. The SA extracts (250 μL) were mixed with an aqueous solution of sodium periodate (0.5% w/v) and sodium m-periodate (0.5% w/v) (250 μL). The mixtures were stirred with a vortex and incubated in the dark in a water bath at controlled temperature (37° C.) for 45 min. Subsequently a 1 M NaOH solution (300 μL) and a 56 mM Na₂SO₃ (200 μL) solution were added to the mixture. The SA oxidized through the effect of the reaction was detected at 382 nm using a microplates reader (Synergy HT, Bio-Tek Instruments Inc., Winooski, Vt., USA). A standard curve for SA was constructed (1-1000 μM) to quantify the compound. The SA concentration was expressed as mg per kg of carrots in dry base (DB). The moisture content in the samples was determined by the method of drying in a stove (AACC 44-15A).

### Example 11

### Identification and Quantification of Shikimic Acid (SA) and Individual Phenolic Compounds (PC) by HPLC-PDA

The methanolic extracts were analyzed by high resolution liquid chromatography, coupled with a photodiode array detector (HPLC-PDA). The system comprised two binary pumps, a self-sampler and photodiode array detector (Waters Corp, Mildford, Mass., USA). The SA and the PC were separated in an inverse phase C18, 4.6 mm×250 mm column, with a particle diameter of 5 μm (Luna, Phenomenex, Torrance, Calif., USA). The mobile phases were water (phase A) and a methanol:water mixture (60:40, phase B) adjusted to a pH of 2.4 with orthophosphoric acid. The mobile phase gradients were 0/100, 3/70, 8/50, 35/30, 40/20, 45/0, 50/0 and 60/100 (min/% phase A) at a constant volumetric flow of 1 mL/min. The data were processed using the Millennium V3.1 program (Waters Corp, Mildford, Mass., USA). SA and individual PC were identified using three procedures: a) comparison of retention time and UV-visible spectrum characteristics for each peak against commercial standards; b) analysis of UV-visible spectrum characteristics and comparison with previous reports; and c) identification by means of elution order on the basis of literature reports in which similar chromatographic conditions to those used in this study were used. The standard curves for different compounds, including shikimic acid (SA), chlorogenic acid (CA), ferulic acid (FA), p-coumaric acid (pCA), protocatechuic acid (PA) and gallic acid (GA) were prepared in a concentration range from 0.5 to 100 μm. The concentration of each was expressed as mg of each individual compound per kg of carrot DB.

1. A process for the overproduction of shikimic acid and phenolic compounds in horticultural crops characterized in that it comprises the stages of:
a) Subjecting a horticultural crop to post-harvest abiotic stress in order to obtain fragments of plant tissue,
b) Incubating the plant tissue obtained in a) for the production and accumulation of shikimic acid and phenolic compounds.

2. The process for the overproduction of shikimic acid and phenolic compounds in horticultural crops as claimed in claim 1, characterized in that a drying stage is optionally included after stage b).

3. The process for the overproduction of shikimic acid and phenolic compounds in horticultural crops as claimed in claim 1, characterized in that a stage of drying and grinding is optionally included after stage b).

4. The process for the overproduction of shikimic acid and phenolic compounds in horticultural crops as claimed in claim 1, characterized in that a stage of glyphosate application is optionally included after stages a) and b).

5. The process for the overproduction of shikimic acid and phenolic compounds in horticultural crops as claimed in claim 4, characterized in that a drying stage is optionally included after stage b).

6. The process for the overproduction of shikimic acid and phenolic compounds in horticultural crops as claimed in claim 4, characterized in that a stage of drying and grinding is optionally included after stage b).

7. The process for the overproduction of shikimic acid and phenolic compounds in horticultural crops as claimed in claim 4, characterized in that a stage of separate recovery, purification and cleaning of shikimic acid and phenolic compounds is included after any one of the stages of incubating, drying or grinding.

8. The process for the overproduction of shikimic acid and phenolic compounds in horticultural crops as claimed in claim 1, characterized in that the horticultural crop in stage a) is preferably carrot tissue (Daucus carota).

9. The process for the overproduction of shikimic acid and phenolic compounds in horticultural crops as claimed in claim 1, characterized in that the abiotic stress in stage a) is wounding stress.

10. The process for the overproduction of shikimic acid and phenolic compounds in horticultural crops as claimed in claim 9, characterized in that the abiotic stress in stage a) is wounding stress combined with one of the following: modified atmospheres, gassing with plant hormones, UV radiation, fluorescent radiation.

11. The process for the overproduction of shikimic acid and phenolic compounds in horticultural crops as claimed in claim 1, characterized in that the wounding stress in stage a) is one of grating, slicing, chopping, mincing and crushing.

12. The process for the overproduction of shikimic acid and phenolic compounds in horticultural crops as claimed in claim 1, characterized in that the wounding stress in stage a) is preferably grating in a diameter range from 2 to 17 mm, preferably 7 mm.

13. The process for the overproduction of shikimic acid and phenolic compounds in horticultural crops as claimed in claim 4, characterized in that in the glyphosate application stage, said glyphosate is in a solution of twice-distilled water or water of superior quality, with a glyphosate concentration range of less than or equal to 482 g/L, with a pH of between 6 and 7.

14. The process for the overproduction of shikimic acid and phenolic compounds in horticultural crops as claimed in claim 4, characterized in that the glyphosate application stage is preferably carried out in a solution having a glyphosate concentration of 482 g/L and a pH of 6.5.

15. The process for the overproduction of shikimic acid and phenolic compounds in horticultural crops as claimed in claim 4, characterized in that the stage of application of the glyphosate solution is carried out by submerging or spraying.

16. The process for the overproduction of shikimic acid and phenolic compounds in horticultural crops as claimed in claim 4, characterized in that the stage of application of the glyphosate solution is preferably carried out by the spraying method.

17. The process for the overproduction of shikimic acid and phenolic compounds in horticultural crops as claimed in claim 4, characterized in that in the stage of applying the glyphosate solution, the grated material is sprayed in a ratio of 1:⅙ to 1:½, namely kg of horticultural crop:L of glyphosate solution.

18. The process for the overproduction of shikimic acid and phenolic compounds in horticultural crops as claimed in claim 4, characterized in that in the stage of applying the glyphosate solution, spraying of the glyphosate solution is preferably carried out with a ratio of 1:⅓, namely kg of horticultural crop:L of glyphosate solution.

19. The process for the overproduction of shikimic acid and phenolic compounds in horticultural crops as claimed in claim 1, characterized in that in incubation stage b) of the stressed horticultural crop, the incubation may be carried out at a temperature of 15-40° C., between 12 and 60 hours storage, at a pressure of between 0.1 and 10 atm, with a relative humidity of between 40 and 100%, in the presence or absence of light.

20. The process for the overproduction of shikimic acid and phenolic compounds in horticultural crops as claimed in claim 19, characterized in that in incubation stage b) of the stressed horticultural crop, the incubation is preferably carried out at 25° C., for 24 hours at atmospheric pressure with a relative humidity of 65%.

21. A nutraceutical grated material obtained by the process of claim 1, characterized in that it has a minimum diameter of 2 mm, a pH in the range from 4 to 6, a moisture content of 15-95%, a shikimic acid concentration of 100 to 500 mg/SA/kg of dry base (ppm) and a phenolic compounds concentration of between 1000 and 3000 mg equivalents of chlorogenic acid/kg dry base (ppm).

22. A nutraceutical grated material obtained by the process of claim 2, characterized in that it has a minimum diameter of 2 mm, a pH in the range from 4 to 6, a moisture content of 2-15%, a shikimic acid concentration of 100 to 500 mg/SA/kg of dry base (ppm), and a phenolic compounds concentration of between 1000 and 3000 mg equivalents of chlorogenic acid/kg dry base (ppm).

23. A dehydrated nutraceutic powder obtained by the process of claim 3, characterized in that it has a particle size range of between 23 and 30% retained on a sieve for particles greater than 0.29 mm, between 10 and 23% retained between 0.10 and 0.29 mm and between 5 and 10% retained on a sieve for particles smaller than 0.10 mm, a pH in the range from 4 to 6, a moisture content of 2-15%, a shikimic acid concentration from 100 to 500 mg/SA/kg of dry base (ppm), and a phenolic compounds concentration of between 1000 and 3000 mg equivalents of chlorogenic acid/kg dry base (ppm).

24. A glyphosated grated material obtained by the process of claim 4, characterized in that it has a minimum diameter of 2 mm, a pH in the range from 3 to 7, a moisture content of 15-95%, a shikimic acid concentration of 2000 to 6000 mg/SA/kg of dry base (ppm), and a phenolic compounds concentration of between 1000 and 3000 mg equivalents of chlorogenic acid/kg dry base (ppm).

25. A dehydrated glyphosated grated material obtained by the process of claim 5, characterized in that it has a minimum diameter of 2 mm, a pH in the range from 3 to 7, a moisture content of 2-15%, a shikimic acid concentration of 2000 to 6000 mg/SA/kg of dry base (ppm), and a phenolic compounds concentration of between 1000 and 3000 mg equivalents of chlorogenic acid/kg dry base (ppm).

26. A glyphosated powder obtained by the process of claim 6, characterized in that it has a particle size range of between 23 and 30% retained on a sieve for particles greater than 0.29 mm, between 10 and 23% retained between 0.10 and 0.29 mm and between 5 and 10% retained on a sieve for particles smaller than 0.10 mm, a pH in the range from 5 to 6, a moisture content of 2-15%, a shikimic acid concentration from 2000 to 6000 mg/SA/kg of dry base (ppm), and a phenolic compounds concentration of between 1000 and 3000 mg equivalents of chlorogenic acid/kg dry base (ppm).
